(19) United States
(12) Patent Application Publication
(10) Pub. No.: US 2002/0094341 A1
(43) Pub. Date: Jul. 18, 2002

(54) SKIN MOISTURIZER COMPOSITIONS CONTAINING A SEBUM CONTROL AGENT

(76) Inventors: LISE W. JORGENSEN, TRUMBULL, CT (US); RICHARD L. MCMANUS, SHELTON, CT (US); ERIC G. SPENGLER, RIDGEFIELD, CT (US)

Correspondence Address: CHARLES J ZELLER, C/O BRISTOL-MYERS SQUIBB COMPANY, 2 BLACHLEY ROAD, STAMFORD, CT, 06922

(21) Appl. No.: 09/428,313

(22) Filed: Oct. 28, 1999

Related U.S. Application Data
(60) Provisional application No. 60/106,810, filed on Nov. 3, 1998.

(51) Int. Cl. A61K 6/00; A61K 7/00

(52) U.S. Cl. 424/401; 514/938

(57) ABSTRACT

The present invention relates to compositions containing a liquid crystal/gel network (LCGN) emulsion thickened with suitable thickening agents containing oil controlling agents.

This is a continuation-in-part application of prior provisional patent application U.S. Serial No. 60/106810 filed Nov. 3, 1999, now abandoned.

### FIELD OF THE INVENTION

The present invention relates to skin moisturizing compositions that allow for effective use of agents that control the level of skin oils on the skin. These compositions comprise a liquid crystal/gel network (LCGN) emulsion and an oil controlling agent.

### BACKGROUND OF THE INVENTION

In JSCC, vol.35, pp.45-57 (January, February 1984), Junginger et al (incorporated herein by reference in its entirety) describe oil-in-water emulsions whose stabilization is provided by a lamellar liquid crystal three-dimensional network. Such emulsions can be referred to as a liquid crystal/gel network or “LCGN”.

In “Secondary droplet emulsion: contribution of liquid crystal formation to physicochemical properties and skin moisturizing effect of cosmetic emulsion” (12^{th} International Congress IFSSC, Paris September 1992, Abstracts, Vol. 1, 117-136), Suzuki et al (incorporated herein by reference in its entirety) describe these oil-in-water emulsions (i.e., LCGNs) as forming superstructures (“secondary droplets”), aggregates of oily droplets coated with liquid crystal lamellae. These authors show that the existence of these superstructures is dependent on the presence of a fatty alcohol. The main qualities of this type of emulsion are the stability with respect to the release of oil and a skin moisturizing effect.

Also known is the article by Dahms in Cosmetics and Toiletries, Vol. 101, November 1986 (incorporated herein by reference in its entirety), which describes emulsions having the same characteristics and, thus, also are LCGNs

Other LCGNs are disclosed in U.S. Pat. Nos. 5,744,062; 5,658,575; 5,674,509; and 5,641,493 incorporated herein by reference in their entirety.

Personal moisturizing compositions are widely used by consumers and must satisfy multiple criteria to be acceptable to consumers. These criteria include relieving and preventing skin dryness (by hydrating the skin or occluding the skin with water-insoluble materials), imparting a soft or smooth feeling to the skin, and mildness. Ideal personal moisturizer compositions should relieve and prevent dry skin, cause little or no irritation, and leave an aesthetically pleasant afterfeel. Another criteria that exists is the need to control surface sebum on the skin. This need is especially felt by the adolescent population. In order to conveniently deliver both moisturization and oil control from a single product, it is highly desirable to develop effective skin moisturizing compositions that also provide a sebum controlling benefit. Typical oil (i.e., sebum)-controlling moisturizers utilize standard emulsion technology, such as oil-in-water technology. The structure of the emulsion and the interaction between the oil (sebum) and the oil(sebum)-controlling ingredients in the standard emulsion technology produces moisturizers with limited oil controlling efficiency. It would be highly desirable to have improved skin moisturizer formulations having improved delivery to the skin of oil or sebum controlling agents.

It is an object of this invention to provide an improved personal moisturizing composition.

It is another object of this invention to provide improved personal moisturizing compositions that provide combined skin moisturizing and oil, i.e., sebum, control.

### SUMMARY OF THE INVENTION

The present invention concerns a skin moisturizer formulation based on a LCGN emulsion. Use of the composition of the invention results in superior delivery and/or superior performance of oil-controlling agents contained in the LCGN emulsion. More particularly, the present invention is directed to a skin moisturizer composition comprising:

(A) a LCGN comprising:

(a) water,

(b) one or more low HLB nonionic co-emulsifiers,

(c) one or more high HLB nonionic primary emulsifiers, and

(B) one or more oil controlling agents, and

(C) optionally, one or more adjuvants, as hereinafter described.

In the composition of the invention the amount of component (A) is typically about 25 to about 99.95 weight %, preferably about 50 to about 95 weight %, more preferably about 75 to about 95 weight %; the amount of component (B) is typically about 0.05 to about 25 weight %, preferably about 0. 10 to about 20 weight %, more preferably about 0.25 to about 10 weight %;

the amount of component (C) is typically about 0 to about 75 weight %, preferably about 5 to about 50 weight %, more preferably about 10 to about 25 weight %. All of the percentages recited herein are based on the total weight of the composition, unless indicated otherwise.

It has been found that successful control of the skin oils (sebum) requires that the skin moisturizer composition of the present invention have a surface tension adapted to disperse the sebum from the skin surface of the user, thereby permitting the oil controlling agent to function in its intended manner. Suitably, the surface tension of the compositions of the present invention is about 25 mN/M or less.

The requisite surface tension of the compositions of the present invention can be achieved through the choice of the primary and co-emulsifier ingredients, the concentrations of these ingredients in the compositions relative to other ingredients, and the relative proportions of these ingredients one to the other, as hereinafter described. In addition, the type and concentration of the oil controlling agent has an effect on the surface tension of the moisturizing products of the present invention.

Thus, the components A(b) and A(c) taken together typically comprise from 0.1 to 20% by weight of the total composition, with the weight ratio of A(b) to A(c) being from about 40:1 to about 1:1. Preferably, the components A(b) and A(c) are present in a combined amount of from about 0.5 to about 15% by weight of the composition. With the preferred lecithin primary emulsifier and the preferred co-emulsifiers disclosed herein, the weight ratio of the components A(b) to A(c) is from about 35:1 to about 10:1, although with other emulsifiers as described herein the ratio is more typically from about 15:1 to 1:1. The concentration of component A(a) is obtained by the difference between the concentration of component A and the combined concentrations of components A(b) and A(c).

### DETAILED DESCRIPTION OF THE INVENTION

Personal moisturizing compositions are widely used by consumers and must satisfy multiple criteria to be acceptable to consumers. These criteria include relieving and preventing skin dryness (by hydrating the skin or occluding the skin with water-insoluble materials), imparting a soft or smooth feeling to the skin, and mildness. Ideal personal moisturizer compositions should relieve and prevent dry skin, cause little or no irritation, and leave an aesthetically pleasant afterfeel. Typically, these compositions are emulsions, either O/W or W/O type. Few examples exist which use LCGNs. The compositions of the present invention have all of these properties.

Separately, a need exists to control surface sebum on skin, especially among adolescents. Current strategies attempt to increase surface tension or use ingredients such as silica to absorb sebum. In order to conveniently deliver both moisturization and oil control from a single product, it is highly desirable to develop effective skin moisturizing compositions that also provide a sebum controlling benefit. The compositions of the invention provide such benefit. Typical oil-controlling moisturizing formulations utilize standard emulsion (oil in water or O/W) technology and, because of the structure of the emulsion and interaction between the oil and oil-controlling ingredients, only limited oil controlling efficiency is realized.

It is therefore an object of the invention to provide improved personal moisturizing compositions which relieve dry skin and which also control oil on the skin (i.e., to provide combined skin moisturizing and oil control).

The present invention concerns a formulation containing a LCGN. Typical oil-controlling moisturizers utilize standard O/W emulsion technology. Although there is some effect on sebum control from standard O/W systems, maximum efficiency is not typically realized. Use of the composition of the invention containing a LCGN, however, helps isolate oil controlling agents from the oil phase in the emulsion. This, in turn, allows maximum availability of the oil controlling agents to control sebum in a superior and more efficient manner.

By the term “liquid crystal/gel network” or LCGN is meant a combination of water, one or more low HLB nonionic co-emulsifiers, and one or more high HLB nonionic primary emulsifiers that, when combined, result in a three-dimensional network consisting of multilamellar bilayers and/or vesicles. The bilayers are oriented in such a way that the hydrocarbon tails are directed towards each other, as are the polar head groups. Hydrophilic molecules reside interlamellarly within the regions between polar head groups. Similarly, lipophilic molecules reside interlamellary within the regions between hydrocarbon tail groups.

It is not desired to be bound by any particular theory or mechanism; however it is believed that the compositions of the invention isolate oil controlling agents from the oil phase of the emulsion, thereby preventing the oil controlling agents from interacting with the other lipophilic components of the emulsion which results in more, or a more effective form, of the oil controlling agents being delivered to oil or sebum on the skin.

The compositions of the invention comprise a LCGN which contains a dispersed, insoluble oil phase. By “dispersed” it is meant that the oil phase can exist as a separate and distinct phase of fine particles or aggregates within the composition. By “insoluble” it is meant that the oil phase has a solubility of less than about 5.0 grams per 100 grams of water at 25° C., preferably less than about 1.0 gram per 100 grams of water at 25° C. In preparing the compositions of the invention, the LCGN is prepared before incorporation of the oil controlling agent(s). In this way the oil controlling agents are isolated from the oil phase already formed in the LCGN.

Surface tension is a physical property of a system (in this case a cosmetic skin moisturizer composition) which is a measurement of the interfacial free energy per unit area. Surface tension measurements for the current invention were made using Kruss K-12 Tensiometer control to 25° C. The compositions of the invention have a surface tension of less than about 25 mN/M, preferably less than about 24 mN/M, more preferably less than about 23mN/m, even more preferably less than about 22 mN/M, even more preferably less than about 21 mN/M, even more preferably less than about 20 mN/M, even more preferably less than about 19 mN/M.

The compositions of the invention are typically prepared by mixing the water soluble or water dispersible ingredients, including the high HLB primary emulsifier, are premixed with water with agitation and heated for a time and temperature sufficient to form a substantially homogenous water phase. Typically, sufficient mixing and heating of the water phase is achieved a temperature of about 65 to about 80° C.; preferably a temperature of about 70 to about 75. In a separate container the lipophilic ingredients, including the low HLB co-emulsifier, are mixed for a time and temperature sufficient to form a substantially homogenous oil phase. Typically, mixing and heating of the oil phase is performed at the same temperatures as for the water phase. A wide variety of oil type and emollient type materials and mixtures of materials are suitable for use in the oil phase of the compositions of the present invention. Typically, the oil phase includes hydrocarbons, fatty acids, phospholipids, fatty alcohols, other emollients, other fatty acid derivatives, cholesterol or cholesterol derivatives, ceramides, vegetable oils, vegetable oil derivatives, and the like. The oil phase is then emulsified into the water phase using a standard mixing apparatus such as a homogenizing mill or the like. The resulting mixture is then cooled until the LCGN is formed. After the LCGN is formed, then the oil controlling agents are added either alone or mixed with a suitable diluent such as glycerin, propylene glycol, and the like. Optional active agents/excipients/adjuvants such as sunscreens, antioxidants, thickeners, fragrances, preservatives, colorants, and the like are preferably added after the LCGN is formed. However in cases where the ingredient cannot be incorporated easily into the LCGN, for example a lipophilic material such as a sunscreen or a hydrophilic material, the optional ingredient can be incorporated into the oil or water phase as the case may be or during homogenation, provided such ingredients are not heat sensitive. In some instances where the ratio of the low HLB coemulsifier to the high HLB primary emulsifiers high, e.g., above 25:1, it is possible to combine with emulsifiers in the oil phase, with further processing as set forth above.

The individual components of the composition of the invention will be discussed individually below.

Emulsifiers

Detailed listings of emulsifying surfactants can be found in McCutcheon's EMULSIFIERS AND DETERGENTS, North America Edition, 1996, McCutcheon Division, MC Publishing Company, incorporated herein by reference.

Nonionic emulsifying surfactants differ from other surfactants by virtue of the absence of charge on or ionization of the molecule.

Low HLB Nonionic Co-emulsifiers

Nonionic co-emulsifiers useful herein include any of the well-known nonionic surface active agents (surfactants) that have an HLB of 6 or less, preferably about 5.5, to 2.5, most preferably about 4.5 to 3.5. The abbreviation “HLB” stands for hydrophilic lipophilic balance. The HLB system is well known in the art and is described in detail in the “The HLB System, A Time-Saving Guide to Emulsifier Selection”, ICI Americas Inc., Aug. 1984, which is incorporated herein by reference.

The identity of the low HLB nonionic co-emulsifier is not particularly limited. Preferably, the co-emulsifier is a solid at room temperature (i.e., about 25° C.). Examples of nonionic low HLB co-emulsifiers capable of forming gel networks include, but are not limited to:

1. Fatty acids and fatty alcohols, including the ethoxylated derivatives thereof.

2. Fatty acid esters, like glyceryl monostearate and similar glyceryl esters, sorbitan fatty acid esters, like sorbitan palmitate, solid polyglyceryl fatty acid esters, and solid methyl glucoside fatty acid esters.

3. Fatty alcohol ethers. Ethoxylated or propoxylated fatty alcohols of 10 to 22 carbon atoms include the lauryl, cetyl, stearyl, isostearyl, oleyl, and cholesterol alcohols having attached thereto from 1 to 50 ethylene oxide or propylene oxide groups.

4. Ether-esters such as fatty acid esters of ethoxylated fatty alcohols.

5. Ethoxylated glycerides, such as ethoxylated glyceryl monostearate

6. Acetoglyceride esters, such as acetylated monoglycerides

7. Ethylene glycol mono and di-fatty acid esters, and diethylene glycol mono- and di-fatty acid esters.

8. Beeswax derivatives, e.g. polyoxyethylene sorbitol beeswax. These are reaction products of beeswax with ethoxylated sorbitol of varying ethylene oxide content, forming a mixture of ether-esters.

9. Lanolin derivatives, e.g., lanolin alcohols, lanolin fatty acids, ethoxylated lanolins.

10. Amides such as fatty acid amides, ethoxylated fatty acid amides, solid fatty acid alkanolamides.

The “fatty” constituents described above unless otherwise stated have a fatty acid residue having from about 8 to 30 carbons, preferably 12 to 22 carbons, and may be saturated or unsaturated.

Other examples of specific nonionic co-emulsifiers include abietic acid, arachidic acid, arachidonic acid, beheneth-5, behenic acid, C18-36 acid, C9-11 pareths, C11-15 pareths, C11-15 pareths oleate, C11-21 pareths, C12-13 pareth-3, C12-15 pareth-2, capric acid, caproic acid, carpylic acid, ceteareths, cetearyl alcohol, ceteths, cetyl alcohol, coconut acid, coconut alcohol, corn acid, cottonseed acid, dodoxynol-5, glyceryl behenate, glyceryl caprate, glyceryl caprylate, glyceryl caprylate/caprate, glyceryl cocoate, glyceryl erucate, glyceryl hydroxystearate, glyceryl isostearate, glyceryl lanolate, glyceryl laurate, glyceryl linoleate, glyceryl myristate, glyceryl oleate, glyceryl palmitate lactate, glyceryl sesquioleate, glyceryl stearate, glyceryl stearate citrate, glyceryl stearate lactate, glycol distearate, glycol hydroxystearate, glycol oleate, glycol ricinoleate, glycol stearate, hydrogenated animal glyceride, hydrogenated coconut acid, hydrogenated cottonseed glyceride, hydrogenated lard glyceride, hydrogenated manhaden acid, hydrogenated palm oil glyceride, hydrogenated tallow glyceride, hydrogenated vegetable glyceride, hydroxystearic acid, isodeceth-5, isolaureth-3, isosteareth-2, isosteareth-3, isostearic acid, laneth-5, lanolin acid, lard glyceride, laureth-5 carboxylic acid, lauric acid, linoleic acid, linolenic acid, linseed acid, methyl glycol sesquioleate, methyl glucose sesquistearate, myreth-3, myreth-4, myristic acid, nonoxynols, nonyl nonoxynol-5, octoxyglyceryl behenate, octoxyglyceryl palmitate, octoxynols, oleic acid, oleths, oleyl alcohol, palmitic acid, palm kernel alcohol, palm oil glyceride, PEG-2 oleate through PEG-5 oleate, pelargonic acid, polyglyceryl-2 diisostearate, polyglyceryl-3 oleate, polyglyceryl-4 oleate, propylene glycol isostearate, propylene glycol hydroxystearate, propylene glycol laurate, propylene glycol myristate, propylene glycol oleate, propylene glycol ricinioleate, propylene glycol stearate, ricinoleic acid, sorbitan diisostearate, sorbitan dioleate, sorbitan isostearate, sorbitan laurate, sorbitan oleate, sorbitan palmitate, sorbitan sesquiisostearate, sorbitan sesquioleate, sorbitan sesquistearate, sorbitan stearate, sorbitan trisostearate, sorbitan trioleate, sorbitan tristearate, soy acid, steareths, stearic acid, sucrose cocoate, sucrose laurate, sucrose stearate, tall oil acid, tallow acid, trideceth-3, undecanoic acid, undecylenic acid, and mixtures thereof. It should be understood that many of the low HLB co-emulsifiers of the present invention also have emollient properties. In such cases the co-emulsifier may be used to partially or completely fulfill the function of the emollient oil/wax component described below.

Numerous other fatty alcohols and glyceryl esters are listed in the CTFA Cosmetic Ingredient Handbook, First Ed., The Cosmetic Toiletry and Fragrance Assn., Inc., Washington, D.C. (1988) at pages 28 and 29, incorporated herein by reference.

Glyceryl esters, fatty acids and fatty alcohols are preferred low HLB nonionic emulsifiers for use herein, and are present in a preferred amount of from about 0.5 to about 15% by total weight of the composition. The most preferred glyceryl ester is glycerol monostearate (glyceryl stearate), or GMS as it is commonly called. While called a monostearate, the monoglycerides, formed by the reaction of glycerin with the appropriate fatty acids, are mixtures of α- and β-monoglycerides, 1,2-and 1,3-diglycerides and some unreacted free fatty acid and glycerin. The surface-active properties are attributed to the monoglyceride, with the diglyceride and triglycerides being practically devoid of surface activity. Many grades of GMS are available in the market place which vary in the grade of stearic acid used or in the ratio of mono-, di-, and triester present.

The fatty acids are preferably long chain fatty acids preferably containing 6 to 60 carbon atoms, preferably 8 to 40 carbon atoms, more preferably 12 to 22 carbon atoms. The fatty acids can be saturated or unsaturated. Specific examples include stearic acid, palmitic acid, lauric acid, myristic acid, isostearic acid, hydroxystearic acid, oleic acid, linolic, ricinoleic acid, arachidic acid, behenic acid, erucic acid and the like.

The fatty alcohols act as emollients as well as help in providing the formation of the LCGN. The fatty alcohols are preferably long chain both alcohols preferably containing 6 to 60 carbon atoms, preferably 8 to 40 carbon atoms, more preferably 14 to 22 carbon atoms, even more preferably 10 to 28 carbon atoms. Lauryl, myristyl, cetyl, hexadecyl, stearyl, isostearyl, hydroxystearyl, oleyl, ricinoleyl, behenyl, erucyl, and 2-octyl dodecanol alcohols are examples of satisfactory fatty alcohols.

The alkoxylated derivatives of the various low HLB co-emulsifiers enumerated above have a degree of alkyoxylation sufficient to provide an HLB value as set forth above.

High HLB Primary Nonionic Emulsifier

Suitable nonionic primary emulsifiers that are useful to form the LCGNs of the present invention have an HLB greater than 6, preferably between about 6.5 to 15, preferably from about 8 to 12. The high HLB primary emulsifiers include ethoxylated fatty alcohols preferably having 3 to 25 moles ethylene oxide; ethoxylated fatty acids preferably having about 4 to 50 moles ethylene oxide; ethoxylated glyceryl fatty acids preferably having about 12 to 30 moles of ethylene oxide; ethoxylated lanolin and ethoxylated lanolin acid esters having about 5 to 75 moles ethylene oxide; ethoxylated alkylphenols having about 3 to 100 moles ethylene oxide; ethoxylated oils having preferably about 8 to 100 moles ethylene oxide; ethoxylated sorbitan and sucrose esters, sorbitan esters preferably having about 8 to 75 moles ethylene oxide; polyoxyethylene/polyoxypropylene block copolymers, especially copolymers having the formula (EO)ₓ (PO)_{y} (EO)ₓ wherein EO and PO represent ethylene oxide and propylene oxide units, respectively; and alkoxylated amines and alkoxylated amides preferably having about 2 to 50 moles ethylene oxide. By the term “fatty” as used to describe acids, alcohols, amines, esters and oils is meant a substituent group or compound as the case may be having from eight to about 30, preferably from 12 to 22, carbon atoms in the group or compound, which may be saturated or unsaturated. In one embodiment the high HLB primary emulsifier is not a sucrose ester when the low HLB co-emulsifier is a sorbitan ester. Of course, the degree of alkoxylation for the alkoxylated materials set forth above must be sufficient to provide an HLB within the prescribed requirements.

Illustrative nonionic primary emulsifiers are identified below using the CTFA nomenclature, which is now in common use in the cosmetic/personal products industry and is employed routinely in ingredient labelling of such products:

C11-15 Pareth -12, -20

Ceteareth -12,-15, -20

Isoceteth-10, -20, -30

Isosteareth -10, -12, -20

Laneth-10, -15, -20

Laureth -10, -12, -20, -25, -30

Nonoxynol -10, -14, -20

Octoxynol -7, -10, -16

Oleth -16

PEG -40, -100 Castor Oil

PEG-5 Cocoate

PEG -20, -150 Dioleate

PEG-10, -15, -30 Glyceryl Oleate

PEG-10, -30 Glyceryl Stearate

PEG -25, -35, -50, Hydrogenated Castor Oil

PEG-10 Lanolate

PEG -10, -20, -50 Lanolin

PEG -10, -14 Laurate

PEG -9, -12, -20, -36 Oleate

PEG-25 Propylene Glycol Stearate

PEG-10, -15, -50 Stearamine

PEG -14, -20, -30, -100 Stearate

PPG-10, -27, -55 Glyceryl Ether

Steareth -13, -16, -21

* In the above listing several numbers follow the CTFA designation in certain instances, as in “Nonoxylnol-4, -6 . . . ” or “Poloxamer 181, 212 . . . ” to indicate several compounds, i.e., Nonoxynol-4, Nonoxynol-6, Poloxamer 181, Poloxamer 212, etc.

Phospholipids are preferred for use herein as the high HLB primary emulsifier and are complex lipids in which one of the primary hydroxyl groups of glycerin is esterified with phosphoric acid which carries an additional ester grouping. The two remaining hydroxyl groups are esterfied with long chain, saturated or unsaturated fatty acids. The most preferred phospholipid is lecithin, which is present in the composition in a preferred amount of from about 0.05 to about 5% by weight of the total composition.

Oil Controlling Agents

The composition of the present include one or more sebum (oil) controlling agents. Such oil controlling agents are sebum absorbing agents or sebum surface tension modifying agents. The amount of oil controlling agent(s) is at a level effective to reduce the amount of sebum on the surface of the skin or disperse the sebum on the skin such that the skin appears less oily to the naked eye.

Sebum absorbing materials used in the compositions of the present invention are selected from the group consisting of starch, calcium silicate, polyethylene, nylon, boran nitride, mica, clays such as bentonite, montmarrillonite and kaolin, zeolite, cyclodextrins, fumed silica, synthetic clays such as polymer powders including natural, synthetic, and semisynthetic cellulose, fluorocarbon resins, polypropylene, modified starches of cellulose acetate, particulate cross-linked hydrophobic acrylate or methacrylate copolymers and mixtures thereof. The hydrophobic polymer is a highly crosslinked polymer, more particularly a highly cross-linked polymethacrylate copolymer. The material is manufactured by the Advanced Polymer Systems, 123 Saginaw Dr., Redwood City, Calif., and sold under the trademarks Polytrap and Microsponge. Sebum absorbing materials preferred for use in the present invention include starch, calcium silicate, boran nitride and mixtures thereof; most preferably starch.

Preferred oil controlling agents that are sebum absorbing materials are starches hydrophobically modified to have a high capacity for loading oils. Such starches can be modified with alkyl or alkenyl substituted dicarboxylic acids. Such materials may contain counter-ions, for example metals such as aluminum. A preferred such material is Natrasorb HFB available from National Starch and Chemical Company, U.S.A, which contains aluminum starch octenylsuccinate. Other suitable materials from National Starch and Chemical include Natrasorb Bath, Dry-Flow PC, Dry-Flow XT, and Dry-Flow Pure.

The surface tension modifying agents are modified protein derivatives that reduce the surface tension of oil. Nonlimiting examples include Vegepol (sodium C8-16 isoalkylsuccinyl soy protein succinate) from Brooks Industries, NJ, and the like.

Optional Ingredients

The following optional ingredients may be incorporated in the composition. Any particular optional ingredient is present in the composition in an amount effective to achieve its functional purpose.

Thickening Agents

The compositions of the present invention may comprise one or more thickening agents. Combined with the LCGN the thickener reduces the surface tension of the system at the oil-water interface. By “water soluble or dispersible” as used herein means that the thickening agents are soluble or dispersible in water at a level of at least about 0.10% by weight at 25° C. It is important that the thickening agent(s) are of the types and in amounts effective to reduce the surface tension of the compositions to be within the required levels. Nonlimiting classes of thickening agents include those selected from the group consisting of carboxylic acid polymers, crosslinked polyacrylate polyacrylamide polymers, polysaccharides, gums, vinyl ether/maleic anhydride copolymers, crosslinked poly(N-vinylpyrrolidones), and mixtures thereof. See U.S. Pat. No. 4,387,107 to Klein et al., issued Jun. 7, 1983 and “Encyclopedia of Polymer and Thickeners for Cosmetics”, R. Y. Lochhead and W. R. Fron, eds., Cosmetics 7 Toiletries, vol. 108, pp. 95-135 (May 1993), which list a variety of thickening or gelling agents, and which are incorporated herein by reference in its entirety. U.S. Pat. No. 5,534,265 also discloses thickening agents (termed gelling agents) the disclosure of which is incorporated herein by reference in its entirety.

Preferred thickening agents for use herein are polyacrylamide polymers, especially nonionic polyacrylamide polymers including substituted branched or unbranched polymers. Monomers to prepare the polymers include acrylamide and methacrylamide which are optionally substituted with one or two alkyl groups, preferably C₁₋₅ alkyl groups. Preferred are acrylate amide and methacrylate amide monomers in which the amide nitrogen is unsubstituted or substituted with one or two C₁₋₃ alkyl groups, e.g., SepiGel 305 from Seppic.

Other thickening agents include polysaccharides such as cellulose, carboxymethyl hydroxyethylcellulose, cellulose esters such as cellulose acetate propionate, hydroxyethyl cellulose, hydroxypropyl cellulose, hydroxypropyl mehtylcellulose, methyl hydroxyethylcellulose, microcrystalline cellulose, sodium cellulose sulfate, celluloses modified wherein the hydroxy groups of the cellulose is hydroxyalkylated to form a hydroxyalkylated cellulose which is then further modified with a C₁₀₋₃₀ straight or branched chain alkyl group through an ether linkage and the like.

Other thickening agents include scleroglucans such as Clearogel CS 11 from Michel Mercier Products, Inc., Mountainside, N.J.

Still other thickening agents are anionic polymers such as acrylates copolymer sold under the tradename Aculyn 22 and 33 by Rohm and Haas, and polyether polyurethane copolymers sold under the tradename Aculyn 44 and Aculyn 46 by Rohm and Haas.

Gums are also preferred thickening agents for use herein. Nonlimiting examples of gums useful as thickening agents include acacia, agar, algin, alginic acid, ammonium alginate, amylopectin, calcium alginate, calcium carrageenan, carnitine, carrageenan, corn starch, dextrin, gelatin, gellan gum, guar gum, guar hydroxyproplytriammonium chloride, hyaluronic acid, hydrated silicas, hydroxypropyl chitosan, hydroxypropyl guar, karaya gum, kelp, locust bean gum, magnesium aluminum silicate, magnesium trisilicate, natto gum, potassium alginate, potassium caeeageenan, propylene glycol alginate, sclerotium gum, tragacanth gum, xanthan gum, and the like.

Emollient Oils/Waxes

The compositions of the present invention also comprise an emollient oil/wax, which emollient is other than components (A)(b) and (A)(c). Emollient oil/waxes, are conventional lipid materials (e.g., oils, fats, and waxes) and silicones and hydrocarbons. As used herein, “emollient oil/wax” refer to a material used to add lubricity to the surface of the skin or to provide a smooth feel to the skin, and does not include a surface active agent as set forth herein. Other emollient oil/waxes are known and can be used herein. See, e.g., CTFA Cosmetic Ingredient Handbook (1^{st} Edition, 1998) at page 26 (Fats and Oils) and page 49 (Waxes). Examples of classes of other emollients include the following:

1. Hydrocarbon oils and waxes. Examples include mineral oil, petrolatum, paraffin, ceresin, ozokerite, microcrystalline wax, polyethylene, and perhydrosqualene.

2. Silicone oils, such as dimethyl polysiloxanes, methylphenyl polysiloxanes, water-soluble and alcohol-soluble silicone glycol copolymers.

3. Triglyceride esters, for example vegetable and animal fats and oils. Examples include castor oil, safflower oil, almond oil, avocado oil, palm oil, sesame oil, and soybean oil.

4. Lanolin, lanolin oil and lanolin wax.

5. Waxes such as beeswax, and vegetable waxes including camauba and candelilla waxes.

6. Alkyl esters of fatty acids. Methyl, isopropyl, and butyl esters of fatty acids are particularly useful herein. Examples of other useful alkyl esters include hexyl laurate, isohexyl laurate, isohexyl palmitate, isopropyl palmitate, decyl oleate, isodecyl oleate, hexadecyl stearate, decyl stearate, isopropyl isostearate, diisopropyl adipate, diiohexyl adipate, dihexyldecyl adipate, diiopropyl sebacate, lauryl lactate, myristyl lactate, and cetyl lactate.

7. Alkenyl esters of fatty acids having 10 to 20 carbon atoms. Examples thereof include oleyl myristate, oleyl stearate, and oleyl oleate.

Optional Adiuvants

The composition of the invention optionally contains other ingredients suitable for use in a skin moisturizer formulation.

Such other ingredients include humectants. A variety of humectants can be employed and can be present at a level of from about 0.1% to about 30%, more preferably about 1% to about 15%, and more preferably about 2% to about 8% of the total formulation weight. These materials include polyhydroxy alcohols such as sorbitol, glycerin, hexanetriol, propylene glycol, hexylene glycol, and the like; polyethylene glycol; sugars and starches; sugar and starch derivatives such as alkoxylated glucose and the like; D-panthenol; hyaluronic acid; lactamide monoethanolamine; acetamide monoethanolamine; 2-pyrrolidone-5-carboxylic acid and mixtures thereof. Especially preferred is glycerin. Other useful humectants include glucisides, lactamide monoethanolamine, and acetamide monoethanolamine. Most preferred are glycerin, propylene glycol, and urea.

Other ingredients optionally present include preservatives such as methylparaben; chealating agents such as tetrasodium ethylene diamine tetraacetic acid, and the like; occlusives such as petrolatum and the like; fragrances; ceramides; colorants; sunscreens, vitamins; antioxidants, antiinflammatories; alpha hydroxy acids; and the like.

The following examples are to illustrate the invention but should not be interpreted as a limitation thereon.

### EXAMPLE 1

Formulations are Prepared According to the Techniques Described Herein and Have the Following Compositions:

| Ingredients | Percent Weight |
|---|---|
| Composition 1 | |
| Water | qs to 100 |
| Capric/Caprilic Triglycerides | 12.00 |
| Behenyl Alcohol (and) Glyceryl Stearate (and) Palmitic | 6.00 |
| Acid (and) Stearic Acid | |
| (and) C12-16 Alcohols (and) | |
| Lecithin | |
| Glycerin | 5.00 |
| Silica | 1.00 |
| Carbomer | 0.40 |
| Triethanolamine | 0.40 |
| DMDM Hydantoin (and) Iodopropynyl Butylcarbamate | 0.10 |
| Fragrance | 0.10 |
| Tetrasodium EDTA | 0.10 |
| Composition 2 | |
| Water | qs to 100 |
| Tricaprin | 5.00 |
| Glycerin | 5.00 |
| Behenyl Alcohol (and) Glyceryl Stearate (and) | 3.00 |
| Palmitic Acid (and) Stearic Acid | |
| (and) C12-16 Alcohols (and) Lecithin | |
| C13-14 Isoparafin (and) Polyacrylamide (and) Laureth-7 | 1.50 |
| Sodium C8-16 Isoalkylsuccinyl Soy Protein Sulfonate | 1.00 |
| Aluminum Starch Octenylsuccinate (and) Acrylates | 1.00 |
| Copolymer (and) Magnesium | |
| Carbonate | |
| Magnesium Aluminum Silicate | 0.75 |
| Xanthan Gum | 0.75 |
| Propylene Glycol (and) Diazolidinyl Urea (and) | 0.50 |
| Methylparaben (and) | |
| Propylparaben | |
| Fragrance | 0.10 |
| Tetrasodium EDTA | 0.10 |
| Composition 3 | |
| Water | qs to 100 |
| Glycerin | 5.00 |
| Octyl Isononanoate | 4.00 |
| Behenyl Alcohol (and) Glyceryl Stearate (and) Palmitic | 2.50 |
| Acid (and) Stearic Acid | |
| (and) C12-16 Alcohols (and) Lecithin | |
| C13-14 Isoparafin (and) Polyacrylamide (and) Laureth-7 | 1.00 |
| Sodium C8-16 Isoalkylsuccinyl Soy Protein Sulfonate | 1.00 |
| Aluminum Starch Octenylsuccinate (and) Acrylates | 1.00 |
| Copolymer (and) Magnesium | |
| Carbonate | |
| Magnesium Aluminum Silicate | 0.50 |
| Xanthan Gum | 0.50 |
| Diazolidinyl Urea (and) Iodopropynyl Butylcarbamate | 0.30 |
| Fragrance | 0.25 |
| Methylparaben | 0.20 |
| Propylparaben | 0.10 |
| Tetrasodium EDTA | 0.10 |
| Composition 4 | |
| Water | qs to 100 |
| Isopropyl Myristate | 8.00 |
| Behenyl Alcohol (and) Glyceryl Stearate (and) | 6.00 |
| Palmitic Acid (and) Stearic Acid | |
| (and) C12-16 Alcohols (and) Lecithin | |
| Glycerin | 4.00 |
| Dipropylene Glycol | 1.00 |
| Polyquaternium 37 (and) Mineral Oil (and) PPG-1 | 1.00 |
| Trideceth-6 | |
| Propylene Glycol (and) Diazolidinyl Urea | 0.50 |
| (and) Methylparaben (and) | |
| Propylparaben | |
| Fragrance | 0.30 |
| Hydroxyethylcellulose | 0.25 |
| Tetrasodium EDTA | 0.10 |
| Urea | 0.10 |
| Composition 5 | |
| Water | qs to 100 |
| Capric/Caprylic Triglyceride | 10.00 |
| Behenyl Alcohol (and) Glyceryl Stearate (and) | 4.50 |
| Palmitic Acid (and) Stearic Acid | |
| (and) C12-16 Alcohols (and) Lecithin | |
| Glycerin | 3.50 |
| Propylene Glycol | 2.00 |
| Sodium C8-16 Isoalkylsuccinyl Soy Protein Sulfonate | 0.50 |
| Silica | 0.50 |
| Acrylates/C10-30 Alkyl Acrylate Crosspolymer | 0.30 |
| Phenoxyethanol (and) Isopropylparaben (and) | 0.30 |
| Isobutylparaben (and) Butylparaben | |
| Fragrance | 0.15 |
| Tetrasodium EDTA | 0.10 |
| Sodium PCA | 0.10 |
| Composition 6 | |
| Water | qs to 100 |
| Octyl Isononanoate | 5.00 |
| Behenyl Alcohol (and) Glyceryl Stearate (and) | 3.00 |
| Palmitic Acid (and) Stearic Acid | |
| (and) C12-16 Alcohols (and) Lecithin | |
| Glycerin | 3.00 |
| C13-14 Isoparafin (and) Polyacrylamide (and) Laureth-7 | 1.00 |
| Sodium C8-16 Isoalkylsuccinyl Soy Protein Sulfonate | 1.00 |
| Aluminum Starch Octenylsuccinate (and) Acrylates | 1.00 |
| Copolymer (and) Magnesium | |
| Carbonate | |
| Magnesium Aluminum Silicate | 0.50 |
| Xanthan Gum | 0.50 |
| Diazolidinyl Urea (and) Iodopropynyl Butylcarbamatae | 0.30 |
| Fragrance | 0.30 |
| Methylparaben | 0.20 |
| Propylparaben | 0.10 |
| Tetrasodium EDTA | 0.10 |
| Composition 7 | |
| Water | qs to 100 |
| Cetyl Octanoate | 4.500 |
| Capric/Caprylic Triglyceride | 4.00 |
| Behenyl Alcohol (and) Glyceryl Stearate (and) | 3.50 |
| Palmitic Acid (and) Stearic Acid | |
| (and) C12-16 Alcohols (and) Lecithin | |
| Glycerin | 3.50 |
| Acrylates Copolymer | 1.00 |
| Sodium C8-16 Isoalkylsuccinyl Soy Protein Sulfonate | 0.50 |
| Carbomer | 0.35 |
| Triethanolamine | 0.35 |
| Phenoxyethanol | 0.35 |
| Fragrance | 0.25 |
| Methylparaben | 0.20 |
| Propylparaben | 0.10 |
| Tetrasodium EDTA | 0.10 |
| Composition 8 | |
| Water | qs to 100 |
| Isopropyl Myristate | 12.00 |
| Behenyl Alcohol (and) Glyceryl Stearate (and) | 4.00 |
| Palmitic Acid (and) Stearic Acid | |
| (and) C12-16 Alcohols (and) Lecithin | |
| Propylene Glycol | 2.50 |
| Glycerin | 2.50 |
| Acrylates Copolymer | 1.00 |
| PVM/MA Decadiene Crosspolymer | 0.50 |
| Xanthan Gum | 0.25 |
| Triethanolamine | 0.25 |
| Fragrance | 0.25 |
| Diazolidinyl Urea (and) Iodopropynyl Butylcarbamate | 0.20 |
| Methylparaben | 0.10 |
| Propylparaben | 0.10 |
| Tetrasodium EDTA | 0.10 |
| Composition 9 | |
| Water | qs to 100 |
| Isostearyl Neopentanoate | 10.00 |
| Behenyl Alcohol (and) Glyceryl Stearate (and) | 4.50 |
| Palmitic Acid (and) Stearic Acid | |
| (and) C12-16 Alcohols (and) Lecithin | |
| Glycerin | 4.50 |
| Silica | 0.50 |
| Acrylates Copolymer | 0.50 |
| PVM/MA Decadiene Crosspolymer | 0.50 |
| Propylene Glycol (and) Diazolidinyl Urea (and) | 0.50 |
| Methylparaben (and) Propylparaben | |
| Triethanolamine | 0.25 |
| Fragrance | 0.25 |
| Tetrasodium EDTA | 0.10 |
| Composition 10 | |
| Water | qs to 100 |
| Octyl Isononanoate | 10.00 |
| Behenyl Alcohol (and) Glyceryl Stearate (and) | 5.00 |
| Palmitic Acid (and) Stearic Acid | |
| (and) C12-16 Alcohols (and) Lecithin | |
| Glycerin | 3.00 |
| C13-14 Isoparafin (and) Polyacrylamide (and) Laureth-7 | 1.00 |
| Sodium C8-16 Isoalkylsuccinyl Soy Protein Sulfonate | 1.00 |
| Aluminum Starch Octenylsuccinate (and) Acrylates | 1.00 |
| Copolymer (and) Magnesium | |
| Carbonate | |
| Magnesium Aluminum Silicate | 0.75 |
| Xanthan Gum | 0.75 |
| Diazolidinyl Urea (and) Iodopropynyl Butylcarbamate | 0.30 |
| Fragrance | 0.30 |
| Methylparaben | 0.10 |
| Propylparaben | 0.10 |
| Tetrasodium EDTA | 0.10 |

### EXAMPLE 2

Process for Preparing a Preferred Formulation of the Invention

In a suitable vessel the water, magnesium aluminum silicate, methylparaben, tetrasodium EDTA, xanthan gum, and polyacrylamide/C₁₃₋₁₄ isoparaffin/laureth-7 are mixed and heated to 75-89° C. with stirring. In a separate vessel the octyl isononanoate, behenyl alcohol, glyceryl stearate, palmitic acid, stearic acid, C₁₂₋₁₆ alcohols, lecithin, and propylparaben are heated to 75-80° C. to form an oil phase. The oil phase is then emulsfied into the water-containing mixture using a homogenizing mill. The batch is then cooled with stirring, during which time the LCGN is formed, to 40° C., then at which time the sodium C₈₋₁₆ isoalkylsuccinyl soy protein sulfonate, premixture of glycerin and aluminum starch octenylsuccinate/acyylates copolymer/magnesium carbonate, preservative, and fragrance are added.

We claim:

1. A skin moisturizer composition comprising:
(A) from about 25 to 99.95% of a liquid crystal gel network comprising:
(a) water,
(b) at least one low HLB nonionic coemulsifier, and
(c) at least one high HLB nonionic primary emulsifier, the concentration of (A)(b) plus (A)(c) being from about 0.1 to about 20%;
(B) From about 0.05 to about 25% of an oil controlling agent, and
(C) From about 0 to about 75% of one or more ingredients selected from the group of active agents, adjuvants and excipients, all percentages being by weight of the total composition,
said composition having a surface tension of 25 mN/M or less.

2. The composition of claim 1 wherein the ratio of the component (A)(b) to component A(c) is from about 40:1 to 1:1.

3. The composition of claim 1 wherein the liquid crystal gel netweork is present in the composition in an amount of from about 50 to 95%, with the combined concentration of component (A)(b) plus (A)(c) being from about 0.5% to about 15%.

4. The composition of claim 3 wherein the ratio of the component (A)(b) to component A(c) is from about 40:1 to 1:1.

5. The composition of claim 1 wherein the oil control agent is selected from the group consisting of starch, hydrophobically modified starch, calcium silicate, clay, polymer powder, cross-linked hydrophobic acrylate or methacrylate copolymers, and modified protein derivatives.

6. The composition of claim 5 wherein the preferred oil controlling agent is selected from aluminum starch octenyl succinate, sodium C₈₋₁₆ isoalkyl succinyl soy protein succinate, and mixtures thereof.

7. The composition of claim 1 wherein the nonionic coemulsifier (A)(b) is selected from the group consisting of fatty acids, fatty alcohols, glyceryl esters, and mixtures thereof.

8. The composition of claim 1 wherein the nonionic primary emulsifier is lecithin.

9. The composition of claim 1 wherein the component (C) is an emollient oil/wax present in an amount of from about 0.1 to 20% by weight of the total composition.

10. The composition of claim 1 wherein the component (C) is a thickening agent present in an amount of from about 0.1 to 10% by total weight of the composition.

11. The composition of claim 10 wherein the thickener is selected from the group consisting of polyacrylamides, xanthan gum and magnesium aluminum silicate.

12. The composition of claim 1 wherein the component (C) is a humectant.

13. The composition of claim 6 wherein the nonionic emulsifier (A)(b) is selected from the group consisting of fatty acids, fatty alcohols, glyceryl acids, and mixtures thereof, wherein the nonionic primary emulsifier (A)(c) is a phospholipid.

14. The composition of claim 13 wherein the surface tension of the composition is less than about 22 mN/M.

15. The composition of claim 13 wherein the surface tension of the composition is less than about 20 mN/M.
